SEQUENCE LISTING (14 sequences)

SEQ ID NO: 1 - DNA, 10111 bp, Artificial Sequence
  promoter (203)..(803)
  -35_signal (726)..(731)
  -10_signal (749)..(755)
  misc_feature (793)..(797): Shine-Dalgarno sequence
catcggtcgatcgacattctatctattctccgtcgccgctgccgtaccagggcttattttgctgctggtttgccgccagacgcttgaatatacacgagtaaatgacaactttatctcccgtaccgcatatccggcaggttatgcctttgccatgtggacactggcggcgggcgtcagcctgttggccgtgtggttactgctattgacgatggacgcgctggatttgacgcacttctctttcctgcctgctctgctggaagtcggggttttagtcgccctttctggcgtcgtgcttggtggtttgctggattatctggcgctacgaaaaacgcatctgacgtaatctgtaaatattatttagctgtagtaatcactcgccgtaattattacggctaaataaacatcagtatcgcttattaatatttgttcttttgtgcggcttagcgtttggaaattattggcgccatttataaattctatttttcttacacgattcagctaatgagtctttattttctcatcacccagttgtcactctaatgataattatttgttgaataattgttttatttcacattggttataccaattgcccgcccagactccttcagcactccccttttgttataacgcccttttgcaacagcttcttaaaatcaacctgatatgttttgccaacatatgtgacctggcagccaaatccaagtaacaggaatttaatcatgtttacagtaatttaaccttcccgtaaaatgcccacacactttaaacgccaccagatcccgtggaattgaggtcgttaaatgagactcaggaaatacaataaaagtttgggatggttgtcattatttgcaggcactgtattgctcagtggctgtaattctgcgctgttagatcccaaaggacagattggtctggagcaacgttcactgatactgacggcatttggcctgatgttgattgtcgttattcccgcaatcttgatggctgttggtttcgcctggaagtaccgtgcgagcaataaagatgctaagtacagcccgaactggtcacactccaataaagtggaagctgtggtctggacggtacctatcttaatcatcatcttccttgcagtactgacctggaaaaccactcacgctcttgagcctagcaagccgctggcacacgacgagaagcccattaccatcgaagtggtttccatggactggaaatggttcttcatctacccggaacagggcattgctaccgtgaatgaaatcgctttcccggcgaacactccggtgtacttcaaagtgacctccaactccgtgatgaactccttcttcattccgcgtctgggtagccagatttatgccatggccggtatgcagactcgcctgcatctgatcgccaacgaacccggcacttatgacggtatctccgccagctacagcggcccgggcttctcaggcatgaagttcaaagctattgcaacaccggatcgcgccgcattcgaccagtgggtcgcaaaagcgaagcagtcgccgaacaccatgtctgacatggctgcgttcgaaaaactggccgcgcctagcgaatacaaccaggtggaatatttctccaacgtgaaaccagacttgtttgccgatgtaattaacaagtttatggctcacggtaagagcatggacatgacccagccagaaggtgagcacagcgcacacgaaggtatggaaggcatggacatgagccacgcggaatccgcccatcaccatcaccatcaccatcaccatcgataaaggggttgaggaagaataaagatgttcggaaaattatcacttgatgcagtcccgttccatgaacctatcgtcatggttacgatcgctggcattattttgggaggtctggcgctcgttggcctgatcacttacttcggtaagtggacctacctgtggaaagagtggctgacctccgtcgaccataaacgcctcggtatcatgtatatcatcgtggcgattgtgatgttgctgcgtggttttgctgacgccattatgatgcgtagccagcaggctcttgcctcggcgggcgaagcgggcttcctgccacctcaccactacgatcagatctttaccgcgcacggcgtgattatgatcttcttcgtagcgatgcctttcgttatcggtctgatgaacctggtggttccgctgcagatcggcgcgcgtgacgttgcgttcccgttcctcaacaacttaagcttctggtttaccgttgttggtgtgattctggttaacgtttctctcggcgtgggcgaatttgcgcagaccggctggctggcctatccaccgctatcgggaatagagtacagtccgggagtcggtgtcgattactggatatggagtctccagctatccggtataggtacgacgcttaccggtatcaacttcttcgttaccattctgaagatgcgcgcaccgggcatgaccatgttcaagatgccagtatttacctgggcatcactgtgcgcgaacgtactgattattgcttccttcccaattctgacggttaccgtcgcgttgttgaccctggatcgctatctgggcacccatttctttaccaacgatatgggtggcaacatgatgatgtacatcaacctgatttgggcctggggccacccggaagtttacatcctgatcctgcctgttttcggtgtgttctccgaaattgcggcaaccttctcgcgtaaacgtctgtttggttatacctcgctggtatgggcaaccgtctgtatcaccgtgctgtcgttcatcgtttggctgcaccacttctttacgatgggtgcgggcgcgaacgtaaacgccttctttggtatcaccacaatgattatcgccatcccgaccggggtgaagatcttcaactggctgttcaccatgtatcagggccgcatcgtgttccattctgcgatgctgtggaccatcggttttatcgtcaccttctcggtgggcgggatgactggcgtgctgctggccgtaccgggcgcggacttcgttctgcataacagcctgttcctgattgcgcacttccataacgtgatcatcggcggcgtggtcttcggctgcttcgcagggatgacctactggtggcctaaagcgttcggtttcaaactgaacgaaacctggggtaaacgcgcgttctggttctggatcatcggcttcttcgttgcctttatgccactgtatgcgctgggcttcatgggcatgacccgtcgtttgagccagcagattgacccgcagttccacaccatgctgatgattgcagccagcggtgcagtactgattgcgctgggtattctctgcctcgttattcagatgtacgtttctattcgcgaccgcgaccagaaccgtgacctgactggcgacccgtggggtggccgtacgctggagtgggcaacctcttccccgcctccgttctataactttgccgtagtgccgcacgttcacgaacgtgatgcattctgggaaatgaaagagaaaggcgaagcgtataaaaagcctgaccactatgaagaaattcatatgccgaaaaacagcggtgcaggtatcgtcattgcagctttctccaccatcttcggtttcgccatgatctggcatatctggtggctggcgattgttggcttcgcaggcatgatcatcacctggatcgtgaagagcttcgacgaggacgtggattactacgtgccggtggcagaaatcgaaaaactggaaaaccagcatttcgatgagattactaaggcagggctgaaaaatggcaactgatactttgacgcacgcgactgcccacgcgcacgaacacgggcaccacgatgcaggcggaaccaaaatcttcggattttggatctacctgatgagcgactgcattctgttctctatcttgtttgctacctatgccgttctggtgaacggcaccgcaggcggcccgacaggtaaggacattttcgaactgccgttcgttctggttgaaactttcttgctgttgttcagctccatcacctacggcatggcggctatcgccatgtacaaaaacaacaaaagccaggttatctcctggctggcgttgacctggttgtttggtgccggatttatcgggatggaaatctatgaattccatcacctgattgttaacggcatgggtccggatcgcagcggcttcctgtcagcgttctttgcgctggtcggcacgcacggtctgcacgtcacttctggtcttatctggatggcggtgctgatggtgcaaatcgcccgtcgcggcctgaccagcactaaccgtacccgcatcatgtgcctgagcctgttctggcacttcctggatgtggtttggatctgtgtgttcactgttgtttatctgatgggggcgatgtaatgagtcattctaccgatcacagcggcgcgtcccatggcagcgtaaaaacctacatgacaggctttatcctgtcgatcattctgacggtgattccgttctggatggtgatgacaggagctgcctctccggccgtaattctgggaacaatcctggcaatggcagtggtacaggttctggtgcatctggtgtgcttcctgcacatgaataccaaatcagatgaaggctggaacatgacggcgtttgtcttcaccgtgctaatcatcgctatcctggttgtaggctccatctggattatgtggaacctcaactacaacatgatgatgcactaagagcggcggttatgatgtttaagcaatacctgcaagtaacgaaaccaggcatcatctttggcaacctgatctcggtgattgggggattcctgctggcctcaaagggcagcattgattatcccctgtttatctacacgctggttggggtgtcactggttgtggcgtcgggttgtgtgtttaacaactacatcgacagggatatcgacagaaagatggaaaggacgaagaatcgggtgctggtgaaaggcctgatctctcctgctgtctcgctggtgtacgccacgttgctgggtattgctggctttatgctgctgtggtttggcgcgaatccgctggcctgctggctgggggtgatgggctttgtggtttatgtcggcgtttatagcctgtacatgaaacgccactctgtctacggcacgttgattggttcgctctccggcgctgcgccgccggtgatcggctactgtgcggtaaccggtgagttcgatagcggcgcagcgatcctgctggctatcttcagcctgtggcagatgcctcactcctatgccatcgccattttccgctttaaggattaccaggcggcaaacattccggtattgccagtggtaaaaggcatttcggtggcgaagaatcacatcacgctgtatatcatcgcctttgccgttgccacgctgatgctctctcttggcggttacgctgggtataaatatctggtggtcgccgcggcggttagcgtctggtggttaggtatggctctgcgcggttataaagttgctgatgacagaatctgggcgcgcaagctgttcggcttctctatcatcgccatcactgccctctcggtgatgatgtccgttgattttatggtaccggactcgcatacgctgctggctgctgtgtggtaacaaaacctctctattaaaaaggtgctacggcaccttttttcttagcattagaaacatatccctctcgaaatatttactaaaaaatccgcatgtttaccccattcgtttgccgctttacactagtcgcgaatttaaaacagaggtggtaatgaacgattataaaatgacgccaggtgagaggcgcgcgacctggggtttagggaccgtattctcgttgcgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctattcgatgataagctgtcaaacatgagcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaaggg

SEQ ID NO: 2 - DNA, 30 bp, Artificial Sequence
  Description of Artificial Sequence PCR primer
ttcacgaacgtgatgcattctgggaaatga

SEQ ID NO: 3 - DNA, 30 bp, Artificial Sequence
  Description of Artificial Sequence PCR primer
ctccttgcatgcgcaagcagaatacggtcc

SEQ ID NO: 4 - DNA, 52 bp, Artificial Sequence
  Description of Artificial Sequence PCR primer
gcacagcggccgcgagctcacgcgtatgtctagataagagcggcggttatga

SEQ ID NO: 5 - DNA, 49 bp, Artificial Sequence
  Description of Artificial Sequence PCR primer
atctagacatacgcgtgagctcgcggccgctgtgcatcatcatgttgta

SEQ ID NO: 6 - protein, 9 aa, Artificial Sequence
  Description of Artificial Sequence  Strep-tag linker
AWRHPQFGG

SEQ ID NO: 7 - DNA, 34 bp, Artificial Sequence
  Description of Artificial Sequence Strep-tag linker, forward strand
ggccgcgcctggcgtcatcctcaattcggtggca

SEQ ID NO: 8 - DNA, 34 bp, Artificial Sequence
  Description of Artificial Sequence Strep-tag linker, reverse strand
cgcgtgccaaattgaggatgacgccgccaggcgc

SEQ ID NO: 9 - protein, 18 aa, Artificial Sequence
  Description of Artificial Sequence Strep-HA-tag linker
AWRHPQFGGYPYDVPDYA

SEQ ID NO: 10 - protein, 9 aa, Artificial Sequence
  Description of Artificial Sequence HA-tag linker
YPYDVPDYA

SEQ ID NO: 11 - DNA, 61 bp, Artificial Sequence
  Description of Artificial Sequence Strep-HA-tag linker, forward strand
ggccgcgcctggcgtcatcctcaattcggtggctacccatacgacgtcccagactacgcta

SEQ ID NO: 12 - DNA, 61 bp, Artificial Sequence
  Description of Artificial Sequence Strep-HA-tag linker, reverse strand
cgcgtagcgtagtctgggacgtcgtatgggtagccaccgaattgaggatgacgccaggcgc

SEQ ID NO: 13 - DNA, 5819 bp, E. coli
  misc_feature (801)..(1748): ORF coding for cyoA (bo3 subunit II)
actactgtcgacattctatctattctccgtcgccgctgccgtaccagggcttattttgctgctggtttgccgccagacgcttgaatatacacgagtaaatgacaactttatctcccgtaccgcatatccggcaggttatgcctttgccatgtggacactggcggcgggcgtcagcctgttggccgtgtggttactgctattgacgatggacgcgctggatttgacgcacttctctttcctgcctgctctgctggaagtcggggttttagtcgccctttctggcgtcgtgcttggtggtttgctggattatctggcgctacgaaaaacgcatctgacgtaatctgtaaatattatttagctgtagtaatcactcgccgtaattattacggctaaataaacatcagtatcgcttattaatatttgttcttttgtgcggcttagcgtttggaaattattggcgccatttataaattctatttttcttacacgattcagctaatgagtctttattttctcatcacccagttgtcactctaatgataattatttgttgaataattgttttatttcacattggttataccaattgcccgcccagactccttcagcactccccttttgttataacgcccttttgcaacagcttcttaaaatcaacctgatatgttttgccaacatatgtgacctggcagccaaatccaagtaacaggaatttaatcatgtttacagtaatttaaccttcccgtaaaatgcccacacactttaaacgccaccagatcccgtggaattgaggtcgttaaatgagactcaggaaatacaataaaagtttgggatggttgtcattatttgcaggcactgtattgctcagtggctgtaattctgcgctgttagatcccaaaggacagattggtctggagcaacgttcactgatactgacggcatttggcctgatgttgattgtcgttattcccgcaatcttgatggctgttggtttcgcctggaagtaccgtgcgagcaataaagatgctaagtacagcccgaactggtcacactccaataaagtggaagctgtggtctggacggtacctatcttaatcatcatcttccttgcagtactgacctggaaaaccactcacgctcttgagcctagcaagccgctggcacacgacgagaagcccattaccatcgaagtggtttccatggactggaaatggttcttcatctacccggaacagggcattgctaccgtgaatgaaatcgctttcccggcgaacactccggtgtacttcaaagtgacctccaactccgtgatgaactccttcttcattccgcgtctgggtagccagatttatgccatggccggtatgcagactcgcctgcatctgatcgccaacgaacccggcacttatgacggtatctccgccagctacagcggcccgggcttctcaggcatgaagttcaaagctattgcaacaccggatcgcgccgcattcgaccagtgggtcgcaaaagcgaagcagtcgccgaacaccatgtctgacatggctgcgttcgaaaaactggccgcgcctagcgaatacaaccaggtggaatatttctccaacgtgaaaccagacttgtttgccgatgtaattaacaagtttatggctcacggtaagagcatggacatgacccagccagaaggtgagcacagcgcacacgaaggtatggaaggcatggacatgagccacgcggaatccgcccattaaaggggttgaggaagaataaagatgttcggaaaattatcacttgatgcagtcccgttccatgaacctatcgtcatggttacgatcgctggcattattttgggaggtctggcgctcgttggcctgatcacttacttcggtaagtggacctacctgtggaaagagtggctgacctccgtcgaccataaacgcctcggtatcatgtatatcatcgtggcgattgtgatgttgctgcgtggttttgctgacgccattatgatgcgtagccagcaggctcttgcctcggcgggcgaagcgggcttcctgccacctcaccactacgatcagatctttaccgcgcacggcgtgattatgatcttcttcgtagcgatgcctttcgttatcggtctgatgaacctggtggttccgctgcagatcggcgcgcgtgacgttgcgttcccgttcctcaacaacttaagcttctggtttaccgttgttggtgtgattctggttaacgtttctctcggcgtgggcgaatttgcgcagaccggctggctggcctatccaccgctatcgggaatagagtacagtccgggagtcggtgtcgattactggatatggagtctccagctatccggtataggtacgacgcttaccggtatcaacttcttcgttaccattctgaagatgcgcgcaccgggcatgaccatgttcaagatgccagtatttacctgggcatcactgtgcgcgaacgtactgattattgcttccttcccaattctgacggttaccgtcgcgttgttgaccctggatcgctatctgggcacccatttctttaccaacgatatgggtggcaacatgatgatgtacatcaacctgatttgggcctggggccacccggaagtttacatcctgatcctgcctgttttcggtgtgttctccgaaattgcggcaaccttctcgcgtaaacgtctgtttggttatacctcgctggtatgggcaaccgtctgtatcaccgtgctgtcgttcatcgtttggctgcaccacttctttacgatgggtgcgggcgcgaacgtaaacgccttctttggtatcaccacaatgattatcgccatcccgaccggggtgaagatcttcaactggctgttcaccatgtatcagggccgcatcgtgttccattctgcgatgctgtggaccatcggttttatcgtcaccttctcggtgggcgggatgactggcgtgctgctggccgtaccgggcgcggacttcgttctgcataacagcctgttcctgattgcgcacttccataacgtgatcatcggcggcgtggtcttcggctgcttcgcagggatgacctactggtggcctaaagcgttcggtttcaaactgaacgaaacctggggtaaacgcgcgttctggttctggatcatcggcttcttcgttgcctttatgccactgtatgcgctgggcttcatgggcatgacccgtcgtttgagccagcagattgacccgcagttccacaccatgctgatgattgcagccagcggtgcagtactgattgcgctgggtattctctgcctcgttattcagatgtacgtttctattcgcgaccgcgaccagaaccgtgacctgactggcgacccgtggggtggccgtacgctggagtgggcaacctcttccccgcctccgttctataactttgccgtagtgccgcacgttcacgaacgtgatgcattctgggaaatgaaagagaaaggcgaagcgtataaaaagcctgaccactatgaagaaattcatatgccgaaaaacagcggtgcaggtatcgtcattgcagctttctccaccatcttcggtttcgccatgatctggcatatctggtggctggcgattgttggcttcgcaggcatgatcatcacctggatcgtgaaaagcttcgacgaggacgtggattactacgtgccggtggcagaaatcgaaaaactggaaaaccagcatttcgatgagattactaaggcagggctgaaaaatggcaactgatactttgacgcacgcgactgcccacgcgcacgaacacgggcaccacgatgcaggcggaaccaaaatcttcggattttggatctacctgatgagcgactgcattctgttctctatcttgtttgctacctatgccgttctggtgaacggcaccgcaggcggcccgacaggtaaggacattttcgaactgccgttcgttctggttgaaactttcttgctgttgttcagctccatcacctacggcatggcggctatcgccatgtacaaaaacaacaaaagccaggttatctcctggctggcgttgacctggttgtttggtgccggatttatcgggatggaaatctatgaattccatcacctgattgttaacggcatgggtccggatcgcagcggcttcctgtcagcgttctttgcgctggtcggcacgcacggtctgcacgtcacttctggtcttatctggatggcggtgctgatggtgcaaatcgcccgtcgcggcctgaccagcactaaccgtacccgcatcatgtgcctgagcctgttctggcacttcctggatgtggtttggatctgtgtgttcactgttgtttatctgatgggggcgatgtaatgagtcattctaccgatcacagcggcgcgtcccatggcagcgtaaaaacctacatgacaggctttatcctgtcgatcattctgacggtgattccgttctggatggtgatgacaggagctgcctctccggccgtaattctgggaacaatcctggcaatggcagtggtacaggttctggtgcatctggtgtgcttcctgcacatgaataccaaatcagatgaaggctggaacatgacggcgtttgtcttcaccgtgctaatcatcgctatcctggttgtaggctccatctggattatgtggaacctcaactacaacatgatgatgcactaagagcggcggttatgatgtttaagcaatacctgcaagtaacgaaaccaggcatcatctttggcaacctgatctcggtgattgggggattcctgctggcctcaaagggcagcattgattatcccctgtttatctacacgctggttggggtgtcactggttgtggcgtcgggttgtgtgtttaacaactacatcgacagggatatcgacagaaagatggaaaggacgaagaatcgggtgctggtgaaaggcctgatctctcctgctgtctcgctggtgtacgccacgttgctgggtattgctggctttatgctgctgtggtttggcgcgaatccgctggcctgctggctgggggtgatgggctttgtggtttatgtcggcgtttatagcctgtacatgaaacgccactctgtctacggcacgttgattggttcgctctccggcgctgcgccgccggtgatcggctactgtgcggtaaccggtgagttcgatagcggcgcagcgatcctgctggctatcttcagcctgtggcagatgcctcactcctatgccatcgccattttccgctttaaggattaccaggcggcaaacattccggtattgccagtggtaaaaggcatttcggtggcgaagaatcacatcacgctgtatatcatcgcctttgccgttgccacgctgatgctctctcttggcggttacgctgggtataaatatctggtggtcgccgcggcggttagcgtctggtggttaggtatggctctgcgcggttataaagttgctgatgacagaatctgggcgcgcaagctgttcggcttctctatcatcgccatcactgccctctcggtgatgatgtccgttgattttatggtaccggactcgcatacgctgctggctgctgtgtggtaacaaaacctctctattaaaaaggtgctacggcaccttttttcttagcattagaaacatatccctctcgaaatatttactaaaaaatccgcatgtttaccccattcgtttgccgctttacactagtcgcgaatttaaaacagaggtggtaatgaacgattataaaatgacgccaggtgagaggcgcgcgacctggggtttagggaccgtattctcgttgcgcatg

SEQ ID NO: 14 - protein, 58 aa, Staphylococcus aureus [sequence omitted]

(19) United States
(12) Patent Application Publication
(10) Pub. No.: US 2004/0191869 A1
(43) Pub. Date: Sep. 30, 2004

(54) Crystallography methods

(76) Inventors: So Iwata, London (GB); Bernadette Byrne, London (GB); Mika Jormakka, London (GB); Jeff Abramson, Uppsala (SE)

Correspondence Address: NIKOLAI & MERSEREAU, P.A., 900 SECOND AVENUE SOUTH, SUITE 820, MINNEAPOLIS, MN, 55402, US

(21) Appl. No.: 10/397,438

(22) Filed: Nov. 5, 2002

Related U.S. Application Data
(63) Continuation of application No. PCT/GB01/02043, filed on May 4, 2001.

(51) Int. Cl. C07H 21/04; C12P 21/04; C12N 15/00; C07K 17/00

(52) U.S. Cl. 435/69.7; 435/320.1; 435/325; 530/350; 536/23.5

(57) ABSTRACT

The present invention provides a recombinant vector comprising, (i) a promoter sequence and (ii) a nucleotide sequence encoding a first protein which, when crystallized with a second protein, is capable of accommodating the second protein in the crystal lattice; said recombinant vector further allowing for the insertion of a further nucleotide sequence encoding a second protein to be located, when crystallized, in the crystal lattice of the first protein. The invention further provides a recombinant vector comprising (i) a promoter sequence and (ii) a nucleotide sequence encoding a first protein which upon crystallization yields crystals having available space in the lattice, so as to allow for the ordered packing of a second protein into the said available space, said recombinant vector further allowing, for the insertion of a further nucleotide sequence encoding a second protein to be accommodated, upon its crystallization, in the said available space in the lattice of the first protein.

### TECHNICAL FIELD

The present invention relates to protein crystallography methods and constructs useful therein, in particular to fusion proteins comprising a first protein and a second protein, whereby the first protein upon crystallization yields crystals having available space in the lattice, so as to allow for the ordered packing of the second protein into the said available space. The invention also relates to methods of crystallization of such a second protein, which e.g. can be a protein, such as a membrane protein, which is otherwise difficult to crystallize. The invention further relates to i.a. recombinant vectors adapted for the expression of a fusion protein as described above.

### BACKGROUND ART

Membrane proteins are involved in a multitude of biological processes; the respiratory chain, photosynthesis, transport of solute molecules and ions and regulating cellular responses to a wide range of biological molecules such as hormones, neurotransmitters and drugs. High resolution structural data has allowed useful insight into the function of a number of integral membrane proteins including the G-protein coupled receptor (GPCR), rhodopsin (Palczewski et al, 2000), the bc₁ complex (Iwata et al 1998), bacteriorhodopsin (Pebay-Peyroula et al, 1997) and cytoclrome c oxidase (Iwata et al, 1995, Tsukihara et al, 1996). Despite these successes the number of resolved membrane protein structures remains extremely small compared with soluble proteins.

Crystallization is necessary to obtain the three-dimensional structure of proteins; it often represents the bottleneck in structure determination. In particular, crystallization of membrane proteins has been difficult (For reviews, see e.g. Durbin & Feher (1996) Annual Review of Physical Chemistry 47, 171-204; and Garavito et al. (1996) Journal of Bioenergetics & Biomembranes 28, 13-27). To date, of approximately 12,000 protein structures deposited in the Brookhaven Protein Database, only 20 are membrane proteins. Furthermore, of these 20, only two are eukaryotic in origin (Iwata et al. (1998) Science 281, 6471; and Tsukihara et al. (1996) Science 272, 1136-1144). These are tiny numbers in light of the fact that it is estimated that 35-40% of the genes within the human genome code for integral membrane or membrane associated proteins. Low levels of endogenous expression, high hydrophobicity and low stability in solution all combine to frustrate the membrane protein crystallographer. In many cases, even if enough pure protein can be obtained, it is impossible to grow crystals suitable for structural analysis if indeed they grow at all. In addition, many proteins fail to produce in E. coli-based expression systems, which have a number of advantages over other expression systems, including low cost and rapid production of high cell densities.

Progress in solving some of these problems has been made using fusion protein technology. Fusion of soluble proteins, such as maltose binding peptide, to the hydrophobic neurotensin receptor, a GPCR, has been shown to increase expression and facilitate purification (Tucker & Grisshammer, 1996). In addition, the crystallisation of cytochrome c oxidase was facilitated by the addition of a monoclonal Fᵥ fragment (Ostermeier et al, 1995). Alternatively, crystallisation in lipidic cubic phase has yielded a high-resolution structure of bacteriorhodopsin (Landau & Rosenbusch, 1996; Pebay-Peyroula et al, 1997). However, widely applicable methods to facilitate the crystallisation of membrane proteins have yet to be described.

Membrane protein crystals often contain a very high solvent content (65-80%; Abramson and Iwata (1999), in Protein Crystallisation, Ed. Terese Bergfors, International University Line pp199-210). This solvent space is filled mainly with detergent micelles and can form very large gaps within the crystal lattice structure, gaps which we have found are large enough to accommodate other proteins.

Carter et al (1994), in Protein and Peptide Lett. 1:175 used a fusion between a six amino acid fragment of a HIV polypeptide and glutathione S-transferase (GST) to allow crystallisation and x-ray analysis of the HIV fragment. In this case, the HIV fragment formed an extension to the GST, forming an integral part of the GST structure.

In Privé (1994) Acta Cryst. D50:375-379 and Privéand Kaback (1996) J Bioenergetics Biomembranes 28:29-34, the cytochrorne b₅₆₂ was cloned within one of the extracellular loops of the LacP protein. This only acted to extend the hydrophilic domains of the highly hydrophobic LacP protein, and the fusion was not suitable for x-ray analysis since it was incapable of producing suitable crystals.

A similar approach was used by Iwata et al (1995) Nature 376:660 where an antibody fragment was used to allow crystallisation of cytochrome c oxidase.

However, the GST, cytochrome b₅₆₂ “carrier” molecules and antibody fragments used previously only provide an extra soluble domain, and none are suitable, for example, by being relatively large enough, to accommodate a second protein or protein fragment within their crystal lattice structure or the available space of the crystal.

The cytochrome bo3 ubiquinol oxidase from E. coli is a member of the heme-copper superfamily of proton-pumping respiratory oxidases (for a review of the heme-copper respiratory oxidases, see García-Horsman et al. (1994) J Bacteriol. 176, 5587-5600). Cytochrome bo3 is a four-subunit respiratory enzyme (FIG. 1) that catalyses the four-electron reduction of O₂ to water and functions as a proton pump (Puustinen et al. (1991) Biochemistry 30, 3936-).

The genes for the cytochrome bo3 subunits are organized within a single operon called the cyo operon (Chepuri et al. (1990) J Biol. Chem. 265, 11185-11192), which is under control of a constitutive, multicistronic promoter (Minacgawa et al. (1990) J. Biol. Chem. 265, 11198-11203). The sequence of the cyo operon and the amino acid sequences of the subunits are disclosed in Chepuri et al. (supra) and in GenBank with accession number J05492 (SEQ ID NO: 13). The cyo operon has been shown to encode five open reading, frames, cyoABCDE (cf. SEQ ID NOS: 1 and 13). The gene products of cyoa, cyob, cvoc and cyod correspond to the cytochrome bo3 subunits II, I, III and IV, respectively. The cyoe gene encodes a protoheme IX farnesyltransferase (Saiki et al. (1993) Biochem. Biophys. Res. Comm. 189, 1491-1497).

Purification of histidine-tagged cytochrome bo3 from E. coli using Ni²⁺ afinity chromatography, is disclosed by Rumbley et al. (1997) Biochimica et Biophysica Acta 1340, 131-142).

### BRIEF DESCRIPTION OF THE DRAWINGS

FIG. 1

Structure of cytochrome bo3. The upper drawing, shows a periplasmic view of the protein, showing the position of subunit IV relative to the other transmembrane spanning helices. The heme group is shown in the upper right hand corner. The lower drawing shows the same protein looking through the membrane.

FIG. 2

Two-dimensional model of subunit IV of cytochrome bo3, showing the three transmembrane spanning domains, intracellular N-terminus and extracellular C-terminus

FIG. 3

Wire model of cytochrome bo3. Subunit IV is shown in the lower left hand corner with the three transmembrane spanning helices labeled. The space available within the crystal lattice (>100 kDa) is shown adjacent.

FIG. 4

The cytochrome bo3 fusion vectors. Expression of the separate subunits is under control of a single multi-cistronic promoter. A multiple cloning site was introduced at the 3′ end of subunit IV and this was used to clone in linkers, to act as bridge sequences, and the proteins of interest.

FIG. 5

Western blot analysis of membranes prepared from GO105 cells expressing pMB908 (UBO only), pMB930, pMB946 and pMB947. Panel A shows a blot probed with anti-his antibody directed against the His9 tag at the C-terminal end of subunit II. This blot clearly shows the expression of all of the modified UBO vector constructs (subunit II is 33 kDa) and that this expression can be localized to the membrane. Panel B shows a blot probed with anti-HA antibody directed against the HA tag in the linker in pMB947. A clear band is seen corresponding to the 17 kDa subunit which is not present for the untagged control.

FIG. 6

Crystals of native cytochrome bo₃, cytochrome bo₃+protein Z and cytochrome bo3+apo A-I. The crystals have different forms, with the native cytocbrome bo₃forming rod like crystals (diffract to 3.5 Å) while the cytochrome bo₃+protein Z crystals (diffract to 6 Å) form as square-plates. Crystals of cytocbrome bo3+apo A-I (diffract to 5 Å) form elongated hexagonal plates.

FIG. 7

Expression of cytochrome bo3+GPCR fusion proteins. This blot was probed with anti-HA antibody directed against the HA tag introduced at the C-terminal end of subunit IV of cytochrome bo3 as a linker sequence. Lane 1 shows undetectable signal for cells only. Lane 2 shows the positive control of bo3 +linker only, which gives a band of 14 kDa corresponding to subunit IV only. Lanes 3 and 4 show the expression of subunit IV+either M1 or CB2 receptor. Faint signals can be seen corresponding to the full-length fusion in both cases.

FIG. 8

Expression of cytochrome bo3+leader peptide/ProW constructs. The blot has been probed with an anti-His antibody and shows the specific (33 kDa) bands corresponding to the His-tagged subunit II of cytochrome bo3 for the control, native bo3, and the two fusion proteins. No band is present for cells only.

FIG. 9

Expression of cytochrome bo3+Apo AI constructs. Each sample was grown to an OD600 of 1.0 prior to harvest. Blot A was probed with Apo AI-specific antibody and shows both whole cells and membranes for the cytochrome bo3+Apo AI constructs. Lane 1 shows the+ve control of a fragment of pure human Apo AI and lane 2 the −ve control of the native cytochrome bo3 construct (pMB908) with no detectable signal. Cytocbrome bo3+Apo AI (pMB1241) yields two specific bands, a full-length product (30 kDa) and a breakdown product (18 kDa). For cytochrome bo3+Strep-tag +Apo AI (pMB1242) only one full-length band is detected, while no specific bands are detected for cytochrome bo3+Strep-Ha-tag, +Apo AI (pMB1243). Cytochrome bo3+protein Z+Apo AI (pMB1244) exhibits the highest level of expression although this fusion protein also undergoes a certain amount of breakdown, yielding a full-length product (50 kDa) and a smaller fragment (14 kDa). The same pattern of expression is seen for both whole cells and membranes demonstrating the localization of the fusion protein to the membrane. Blot B (lanes 1-8) was probed with anti-His antibody specific for the His9 tag at the C-terminal end of subunit II. No signal is detected for the −ve control of cells only. The +ve control of cytochrome bo3 shows a distinct band corresponding to subunit II (33 kDa). All the other constructs yield similar bands except pMB1243 (not shown on this blot) and interestingly all seem to exhibit higher expression levels than the control sample. Lane 9 of blot B shows pMB1244 probed with peroxidase anti-peroxidase specific to protein Z. Interestingly, it is only possible to detect the breakdown product using this conjugate, no full length protein can be observed.

FIG. 10

Effects of arabinose concentration on the expression of cytochrome bo3+pBAD. The blot has been probed with anti-His antibody specific for the His-tag, at the C-terminal end of subunit II. The cells containing the cytochrome bo3+pBAD were grown to the mid-log phase and then induced with increasing concentrations of arabinose as detailed on the gel. Cells were harvested 4 h post-induction. No signals are detected for either GL101 cells only or for the uninduced control while a strong band is detected for the +ve control, cytochrome bo3 under control of the native constitutive promoter. The production of cytochrome bo3 under control of the inducible promoter showed a clear dose response relationship with increasing concentrations of arabinose yielding increasing levels of detectable protein up to 0.2% arabinose. The amount of protein expressed at 0.2% arabinose was much higher than that under control of the native constitutive promoter. However, arabinose concentrations higher than 0.2% had no further increase on the expression level (results not shown). The results are the same for whole cells and membranes deinonstrating the localization of the protein to the membrane.

FIG. 11

Effects of induction time on the expression of cytochrome bo3+pBAD promoter. The blot is probed with anti-His antibody specific to the His-tag at the C-terminal end of subunit II. A distinct band is seen for the control of native bo3 while no signal is detected for the uninduced cytochrome bo3+pBAD. After the cells had reached the mid-log phase protein production was induced with 0.2% arabinose and the cells incubated at +37° C. for the times indicated on the gel prior to sampling. A strong signal is detected one hour post-induction with a slight increase thereafter to 3 h after induction. After this time there is a steady drop in the detectable expression of the tagged protein, to about a 50% reduction after the cells had been incubated overnight (18 h). It is unclear whether this loss in detectable protein is a result of degradation of the whole cytochrome bo3 molecule or specific proteolysis of the His-tag.

FIG. 12

Crystal packing for cytocbrome bo₃+apo A-I. Cytochrome bo₃ is shown in white/pale grey and apo A-I in grey.

### DISCLOSURE OF THE INVENTION

In a first aspect, this invention provides a recombinant vector comprising (i) a promoter sequence and (ii) a nucleotide sequence encoding a first protein which is a membrane protein or multisubunit protein and which, when crystallized with a second protein, is capable of accommodating the second protein in the crystal lattice; said recombinant vector further allowing for the insertion of a further nucleotide sequence encoding a second protein to be located, when crystallized, in the crystal lattice of the first protein wherein the resulting crystal lattice is capable of diffracting x-rays.

By “crystal lattice” we mean the crystal lattice produced by the first protein.

It will be appreciated however, that the crystal lattice is formed by the protein which makes and maintains most of the crystal contacts within the lattice, and that the crystal lattice itself may be altered by the presence of a second protein. Such an altered crystal lattice is included in our definition of “crystal lattice”.

A second aspect of the invention provides a recombinant vector comprising (i) a promoter sequence and (ii) a nucleotide sequence encoding a first protein which is a membrane protein or multisubunit protein and which upon crystallization yields crystals having available space in the lattice, so as to allow for the ordered packing of a second protein into the said available space; said recombinant vector further allowing for the insertion of a further nucleotide sequence encoding a second protein to be accommodated, upon its crystallization, in the said available space in the lattice of the first protein wherein the resulting crystal lattice is capable of diffracting x-rays.

The said “space” may be utilized to force a second “target” protein to pack in an ordered manner into the crystal lattice of the first protein, which is used as a “scaffold” molecule. In addition, fusing a second protein to a first protein may facilitate the expression, folding stability in E. coli of the said second protein. The recombinant vectors according to the invention thus provide a template for facilitated/improved expression, purification, crystallization and subsequent structure determination of proteins.

In a preferred embodiment of the first and second aspects, the crystal produced is one capable of diffracting X-rays or is one that is useful for X-ray analysis. As is explained below, the degree or resolution to which a crystal diffracts may be determined by the crystallization conditions. Preferably the crystal lattice produced by the first and second proteins is capable of diffracting x-rays to a resolution of at least 6 Å, 5 Å, more preferably at least 4 Å, 3.5 Å, 3.25 Å or 3 521 . Still more preferably, the crystal lattice can diffract x-rays to a resolution of more than 2.75 Å or 2.5 Å.

The second protein can be expressed together with the first protein as a fusion protein, or alternatively the second protein can be expressed separately and positioned in the available space of the first protein by means of non-covalent interactions. The specificity and affinity necessary for this binding may be achieved by fusing protein tags or domains having such affinity for each other to the first and second protein, respectively. When the expression of a fusion protein is desired, the recombinant vector of the invention is adapted to allow for the insertion of at least one further nucleotide sequence, in particular a sequence encoding the second protein to be accommodated, upon its crystallization, in the said available space in the lattice of the first protein. It will be appreciated that the location of the nucleotide coding sequence encoding the second protein may be in one or more positions relative to the sequence encoding the first protein. In other words, the sequences encoding the first and second proteins may be in any order which provides for the second protein to be accommodated, upon its crystallisation, in the crystal lattice of the first protein. Hence, the sequences may be consecutive in any order, either contiguous or separated by a further sequence, or they may be non-consecutive, for example, the sequence of one protein may be inserted into the coding sequence of the other protein. Where the sequence of one protein is inserted into the coding sequence of the other protein, it is preferred if this is done such that the reading frame of the “other protein” is not changed.

The recombinant vector according to the invention comprises a promoter sequence operably linked to the structural gene(s) and is capable of mediating the expression of the said first protein or the said fusion protein. The term “operably linked” as used herein means that the promoter is functionally linked to a structural gene in the proper position to express the structural gene under control of the promoter.

The skilled person will be able to determine which proteins are suitable for use as the said “first protein” based on the crystal lattice structure of said protein, the size of available cavities in this structure and the positions of sites available for attaching fusion partners. In determining whether a protein is suitable for use as the said “first protein”, it will be appreciated that the size of available cavities or space in the crystal structure of the first protein when crystallized in the absence of a second protein may not be strictly limiting in practice. The first protein may assist in crystallization of the second protein even if the crystal space generated by the first protein and which is available to accommodate the second protein is not identical in the presence and absence of the second protein. This flexibility is sufficient so as to allow the first protein to modify its space group when crystallizing. Thus, the space group of the crystals of the first protein alone may differ from that of crystals of the first and second proteins when together.

Hence, it will be appreciated that the crystal lattice of the first protein useful in the present invention, when crystallised in the absence of a second protein, may have spaces or gaps which are solvent filled and which are smaller than the size of the second protein to be crystallised. Preferably the gaps or spaces in the crystal lattice of the first protein are at least the same size as the second protein.

The first protein may be any protein which is suitable for accommodating the second protein in its crystal lattice. Hence, the first protein may be a soluble protein, including a soluble multisubunit protein, or it may be a membrane protein including a membrane-associated protein or an integral membrane protein. Where the first protein is a multisubunit protein it may have any number of subunits, including 2, 3, 4, 5 or 6 or more. Preferably, where the first protein is a multisubunit protein, it is not an antibody or antibody fragment such as an Fv molecule or Fab-like molecule. Where the first protein is an integral membrane protein, it may have one transmembrane domain, or two or three or more transmembrane domains ie, it may be a polytopic membrane protein). Preferably the first protein is an integral membrane protein, and more preferably it has one transmembrane domain. Still more preferably the first protein has 2 or 3 or 4 or 6 or 7 or 12 transmembrane domains.

The first protein may be any size which, when crystallised, is capable of the required accommodation of the second protein. Preferably the first protein is bigger than 10 amino acids in total, more preferably bigger than 25, 50, 75 or 100 amino acids in total. Still more preferably, the first protein is bigger than 150 aa, 200 aa, 250 aa, 300 aa, 400 aa, 500 aa, 600 aa, 700 aa, 800 aa or 1000 aa in total length. By “total length” we mean the total number of amino acids in the first protein, including all component subunits where the first protein is a multisubunit protein.

It will be appreciated that in order to crystallise the first and second proteins together in a crystal lattice of a quality suitable for x-ray diffraction, it may be necessary to conduct a screen for suitable and optimal conditions for crystallisation. Suitable screens are discussed in more detail below. Such screening is routine in the art of crystallisation. A way of determining if a crystal is capable of diffracting x-rays is to observe the diffraction pattern.

Of the first and the second protein useful in the present invention, it may be determined which protein constitutes the crystal lattice by determining which protein contributes the greater proportion of crystal contacts which are made and maintained within the lattice.

In preferred forms of the first and second aspects of the invention, the said first protein is E. coli cytochrome bo3 or E. coli fumarate reductase, or variants thereof. As shown in FIG. 3, the crystals of cytochrome bo3 provide space for additional proteins at the C-terminal end of cytochrome bo3 subunit IV. The term “variants thereof”, as used herein, is intended to mean e.g. polypeptides carrying modifications like substitutions, small deletions, insertions or inversions, which polypeptides nevertheless have substantially the functional properties of E. coli cytochrome bo3 or E. coli fumarate reductase. It should be emphasized that the term “functional properties”, as used in this context, does not refer to biological activity, but rather to the structural capability to assist in crystallization of the second protein, for example to harbor a second protein in its “available space” in order to facilitate crystallization of the said second protein.

The term “available space” is not to be construed as referring solely to a “cavity” or gap” within the crystal lattice of a said first protein. Rather, the available space also comprises solvent channels in the said crystal lattice. For instance, in the bo3 oxidase crystal (cf. FIG. 12), big “gaps” are repeating, which gaps are not isolated but connected by solvent channels. In the bo3-Apo AI crystal (FIG. 12), Apo AI is not staying in one gap, rather it extends through multiple gaps connected by solvent channels.

The second protein may be any second protein which is capable of being accommodated in the crystal lattice of the first protein. In a preferred embodiment, the second protein is smaller than the first protein. By “smaller” we include the meaning that the second protein has a lower molecular weight than the first protein. A lower molecular weight is any mass which is less than that of the first protein. Preferably, the molecular weight of the second protein is at least 1 kDa, 5 kDa, 10 kDa, 15 kDa or 20 kDa lower than that of the first protein. More preferably the molecular weight of the second protein is smaller than that of the first protein by at least about 25 kDa, 35 kDa, 45 kDa or 55 kDa or more. Similarly, it is preferred if the second protein is not bigger than 150 kDa a, more preferably no bigger than 125 kDa, 110 kDa, 100 kDa or 90 kDa. The size of the second protein may be less than 80 kDa or 70 kDa.

Hence, it will be appreciated that the term “available space” is one which indicates the space in a crystal lattice of a first protein, which space is not occupied by the first protein. This space therefore may be occupied by a second protein or by solvent molecules and still be referred to as “available space”.

Furthermore, the term “available space” as defined above is not to be construed as referring only to a fixed volume within the crystal lattice of a said first protein in the absence of the second protein. Instead, the available space is one which is flexible and may alter in size and/or shape according to the nature of the second protein. In other words, the crystal group produced by crystallization of a first protein compared with that produced by crystallization of a first and second protein may not be the same.

Hence, in one embodiment of the first and second aspects of the invention, the crystal space group of the first protein on its own (ie, when the first protein is crystallized in the absence of the second protein) may be different to that obtained by crystallization in the presence of, or fused to, the second protein.

It will be appreciated that the second protein need not be fused to the first protein to allow its crystallization, and the first and second proteins may be, for example, produced as a fusion, cleaved, then crystallized.

According to another preferred embodiment of the first two aspects of the invention, the first and second proteins are fused to each other. The fusion may be “direct” such that the amino acid sequences of the two proteins are contiguous, or “indirect”, where the amino acid sequences of the two proteins are joined via a linking polypeptide sequence or sequences.

In a further preferred embodiment of the first and second aspects of the invention, the first protein is a multisubunit protein. The subunits may be held together by covalent or non-covalent bonds. An example of a multisubunit protein is E. coli cytochrome bo3.

Preferably, the first protein is one which, when crystallized in the absence of a second protein, its crystal lattice has solvent filled gaps of a suitable size for accommodating a second protein within the crystal lattice, whether or not the original space group of the first protein is maintained by accommodating the second protein. Hence, it will be appreciated that the first protein may be a soluble protein.

When the “first protein” is E. coli cytochrome bo3, the nucleotide sequence to be included in the recombinant vector of the invention is preferably selected from

(a) the polypeptide coding regions of the nucleotide sequence shown as SEQ ID NO: 13;

(b) nucleotide sequences, which e.g. can be at least 90% or 95% homologous, with the nucleotide sequence shown as SEQ ID NO. 13 in the Sequence Listing, and which are capable of hybridizing, under stringent hybridization conditions, to a nucleotide sequence complementary with the polypeptide coding regions of the nucleotide sequence as defined in (a); and

(c) other nucleic acid sequences encoding the same amino acid sequences as those defined in (a) or (b). Numerous such nucleotide sequences may be designed due to the degeneracy of the genetic code.

The term “stringent hybridization conditions” is known in the art from standard protocols (e.g. Ausubel et al., spra) and could be understood as e.g. hybridization to filter-bound DNA in 0.5 M NaHPO₄, 7% sodium dodecyl sulfate (SDS), 1 mM EDTA at +65 C., and washing in 0.1×SSC/0.1% SDS at+68 C.

It should thus be understood that the nucleotide sequence coding for cytochrome bo3, or a suitable variant thereof, is not limited strictly to the sequence shown as SEQ ID NO: 13. Rather this sequence is represented in DNA molecules carrying modifications like substitutions, small deletions, insertions or inversions, which nevertheless encode polypeptides having substantially the functional properties of E. coli cytochrome bo3. As mentioned above, the term “functional properties”, does not in this context refer to biological activity, but rather to the structural capability to harbor a second protein in its “available space” in order to facilitate crystallization of the said second protein.

The promoter sequence to be included in the recombinant vector may be the one naturally associated with the DNA sequence encoding the first protein, or of another origin. When the said first protein is E. coli cytochrome bo3, the said promoter sequence could essentially comprise the cytochrome bo3 promoter sequence shown as positions 203 through 803 in SEQ NO: 1. Alternatively, the said promoter could be an inducible promoter, such as the promoter pBAD (1Invitrogen Corp., CA, USA) as shown in Example 7, below.

In a preferred form of the invention, the recombinant vector can further comprise a nucleotide sequence encoding a linker amino acid sequence, facilitating for the said first and second proteins to be expressed as a fusion protein. The ideal linker should have the appropriate length and flexibility so as to allow the second protein to be positioned in the available space of the crystal lattice, it should not form hydrophobic interactions with lipophilic structures such as host cell membranes or the protein core, it should not affect, in a negative way, the expression, translocation and folding of the fusion protein, it should not inhibit the functions of the first or second proteins and should be stable in the host cell and during purification. In addition, the linker may comprise sequences useful for the detection and/or purification of the fusion protein by means of affinity methods, especially when useful antibodies are unavailable. The said linker amino acid sequence can e.g. be a Strep-tag having an amino acid sequence shown as SEQ ID NO: 6 or a Strep-HA-tag, having an amino acid sequence shown as SEQ ID NO: 9. Preferably, the said linker amino acid sequence will be adapted to facilitate, upon expression of the said first and second proteins, for the said second protein to be positioned in the said available space in the lattice of the first protein. Thus, when the said first protein is E. coli cytochrome bo3, the said nucleotide sequence coding for a linker amino acid sequence can preferably be positioned at the 3′-end of the nucleotide sequence coding for E. coli cytochrome bo3 subunit IV.

As shown in Example 3, below, the recombinant vector according to the invention can in addition comprise a nucleotide sequence encoding a “protein Z” polypeptide having essentially an amino acid sequence shown as SEQ ID NO: 14. Since protein Z is a highly soluble and stable protein domain, its presence may facilitate the expression, solubilization, purification and crystallisation of the fusion protein.

The recombinant vector according to the invention may in addition comprise a nucleotide sequence encoding an affinity tag, e.g. a His-tag, useful for detection and or purification of the expressed protein(s). An affinity tag is a protein sequence that provides a defined, strong, and highly specific non-covalent binding interaction to a ligand or another protein sequence or domain. The presence of the affinity tag in the expressed protein allows the detection and/or purification of said protein on the basis of a reversible interaction between said affinity tag and its specific ligand, said specific ligand being attached to an easily detected chemical entity e.g. a fluorophore or an enzyme) or chromatographic matrix, respectively. When the first protein is E. coli cytochrome bo3, the affinity tag could e.g. be attached to the nucleotide sequence encoding subunit II of E. coli cytochrome bo3 (cf. positions 1746 through 1772 in SEQ ID NO: 1).

In a further important aspect of the invention, the recombinant vector defined above further comprises a nucleotide sequence encoding the said “second” protein. Preferably, the second protein is one as described above. When cytochrome bo3 is the first protein, it is estimated that the “cavity” in the cytochrome bo3 crystal (cf. FIG. 3) can harbor a protein having a molecular mass up to approximately 100 kDa.

As explained above, the presence of a second protein in a crystal of the first protein may alter the space group of the first protein from that which is formed when the first protein is crystallized in the absence of the second protein. Hence, it will be appreciated that the “cavity” in the cytochrome bo3 crystal may in fact be capable of harbouring a protein larger than 100 kDa. Hence, due to the flexibility of the crystal space group, the predicted size of the “cavity” is not to be considered limiting in the choice of second protein.

Consequently, at least when cytochrome bo3 is the first protein, the said second protein preferably has a molecular mass below 100 kDa, such as below 75 kDa, below 60 kDa or below 50 kDa. The skilled person will be able to determine the possible size of the second protein, depending on the crystal lattice structure and available positions for the attachment of fusion partners in the first protein to be used. Furthermore, the second protein must be expressed in the system used in a correctly folded form and be able to translocate within the host cell in a manner consistent with the intended subcellular location and orientation of the fusion protein. In addition, the function of the second protein should be maintained when expressed in the system used, so as to allow a functional assay to be performed, demonstrating that the second protein is in its native or native-like form.

In a preferred embodiment of the invention, the second protein is a membrane protein, and more preferably, it is an integral membrane protein. Such an integral membrane protein may have any number of transmembrane domains, ranging from one to twelve or more.

Preferably, the second protein has a lower molecular weight compared to the first protein.

Included in the invention is also a cultured host cell, e.g. an E. coli cell, harboring a recombinant vector according, to the invention, in particular a recombinant vector comprising a nucleotide sequence encoding for a said second protein.

A further aspect of the invention is a process for the production of a fusion protein comprising culturing a host cell as defined above, under conditions whereby the said fusion protein is produced, and recovering the said fusion protein. A fusion protein obtained, or obtainable by this process is included in the invention.

In the case that the recombinant protein is a multisubunit complex kept together by non-covalent forces the nucleic acid sequences encoding the individual subunits of said complex may be introduced into the host organism by use of more than one vector, each vector encoding one or more of these subunits.

A further aspect of the invention provides a fusion protein comprising (i) a first protein which is a membrane protein or multisubunit protein and which, when crystallized with a second protein, is capable of accommodating the second protein in the crystal lattice and (ii) a second protein to be located, when crystallized, in the crystal lattice of the first protein wherein the resulting crystal lattice is capable of diffracting x-rays.

In yet another aspect, the invention provides a fusion protein comprising (i) a first protein which is a membrane protein or multisubunit protein and which upon crystallization yields crystals having available space in the lattice, so as to allow for the ordered packing of a second protein into the said available space; and (ii) a second protein to be accommodated, upon crystallization, in the said available space wherein the resulting crystal lattice is capable of diffracting x-rays.

When the said first protein is E. coli cytochrome bo3, the said second protein is preferably attached to subunit IV of E. coli cytochrome bo3.

In a preferred embodiment, either or both of the proteins comprised in the fusion proteins of the invention are membrane proteins. In other words, either the first or second proteins, or both of them, are membrane proteins.

By “membrane protein” we include membrane associated proteins, membrane inserted proteins (such as those which possess a hydrophobic domain which is resident within a membrane but may not completely span the membrane bilayer) and integral membrane proteins where the protein possesses at least one transmembrane domain which spans the membrane bilayer, such as single spanning integral membrane proteins and polytopic membrane proteins. Preferably, the membrane protein is an integral membrane protein.

More preferably, the first protein is one as defined above in relation to the first and second aspects of the invention.

In a further aspect, the invention provides a method for crystallization of a protein, comprising (i) obtaining a fusion protein comprising (I) a first protein, which is a membrane protein or multisubunit protein and which upon crystallization yields crystals having available space in the lattice, so as to facilitate crystallization of a second protein; and (II) the said (second) protein to be crystallized; and (ii) crystallizing the said fusion protein wherein the resulting crystal is capable of diffracting x-rays.

The said fusion protein could preferably be obtained by the processes described above, in particular by expression of the recombinant vectors according to the invention.

Preferably the first protein is a membrane protein, and more preferably it is an integral membrane protein.

A still further aspect of the invention provides a method for crystallization of a protein, comprising (i) obtaining a first protein which is an integral membrane protein and which upon crystallization yields crystals having available space in the lattice, so as to facilitate crystallization of a second protein; and (ii) obtaining the second protein to be crystallized; and (iii) crystallising both the said proteins together wherein the resulting crystal is capable of diffracting x-rays.

The first and second proteins of this aspect could be produced as a fusion protein, preferably obtained by the processes described above, in particular by expression of the recombinant vectors according to the invention.

In an embodiment of this aspect of the invention, the proteins could be obtained by expressing the first and second proteins in two separate expression systems and fusing them after purification, either by simply mixing the two purified protein samples or by soaking that second protein into crystals of the first protein. It will be appreciated that in order to introduce the second protein into the solvent gap of the crystals of the first protein by soaking it is necessary to have a means of targeting the second protein to the precise location within the crystal lattice of the first protein, i.e. some form of protein-protein interaction. This can be achieved using high affinity domains engineered into suitable sites within the proteins, which when a suitable concentration of the second protein is added to the crystals of the first protein, allows the proteins to form a complex based on the affinity of the two domains.

Preferably, the first protein useful in the methods of crystallisation according to the present invention is as defined above in respect of the recombinant vectors of the invention.

In a preferred embodiment of the crystallisation methods of the invention, the second protein is as defined according to the first or second aspects of the invention.

The second protein may be a soluble protein, a membrane associated protein or an integral membrane protein. The method is particularly useful where the second protein is an integral membrane protein. Hence it is preferred if the second protein is an integral membrane protein.

In a preferred embodiment of the crystallisation method aspects of the invention, the method further comprises a step wherein at least two detergents are screened in the crystal growth conditions to identify which one optimises the growth and/or diffraction of the resulting crystals. It is known that detergent selection is important to obtain well diffracting crystals. In the case where cytochrome bo3 is the first protein, a wide range of detergents are tolerated, providing a broad choice of detergent in order to maximise the resolving ability of the crystals produced.

Suitable detergents for screening include all detergents of the C7-C9 range.

Preferably, one of the detergents screened is octylglucoside.

Optimal growth of the crystals is that which gives a smaller number of crystals, preferably a single crystal, which are large. Preferably the resulting crystal is assessed by its diffraction pattern, with those crystals which produce a diffraction pattern being preferred to those which do not.

In a further preferred embodiment of these crystallisation method aspects, the pH of crystallisation is optimised for crystal growth. The pH screening may be performed between a range of pH 6-8. Typically, an initial screen to optimise pH of crystallisation may test pH values of 6, 6.5, 7, 7.5 and 8. Preferably, this embodiment comprises a further screen for an optimised pH wherein the optimal pH value identified by the initial pH screen is tested to a finer degree. For example, where pH 6.5 is identified as optimal in the first screen, a subsequent pH screen may test pH values of 6.3, 6.4, 6.5, 6.6, 6.7 and 6.8.

According to a yet further preferred embodiment, the optimisation of crystallisation pH is performed in addition to a screen to identify a detergent which optimises the growth of well-diffracting crystals.

When the second protein is a hydrophobic protein, such as an integral membrane protein, an altered pH and a higher PEG concentration (relative to the conditions employed for crystallization of the first protein alone) may be necessary.

PEG acts as a precipitant in crystallisation, and acts to alter the protein-solvent or protein-protein contacts so that the protein molecules precipitate out of solution, preferably as ordered crystals. Other precipitants are known to be useful in crystallisation, including, for example, ammonium sulphate and 2-methyl-2,4-pentanediol (MPD). Alternative precipitants are give in Bergfors (1999) in Protein Crystallization Ed. Terese Bergfors, International University Line pp 41-50.

It will be appreciated that the crystal space group of the first protein alone (ie, in the absence of the second protein) may be different to that obtained by crystallization of the first protein with the second protein.

An additional aspect of the invention provides a method of obtaining structural data on a protein of interest comprising the steps of

(i) obtaining the protein of interest;

(ii) crystallising said protein in the crystal lattice of another protein, which crystal lattice is able to accommodate the protein of interest; and

(iii) obtaining x-ray diffraction data from the crystal produced in step (ii).

The crystallisation method used in step (ii) may be any suitable method. Preferably it is a method according to the present invention as described above.

The protein of interest may be obtained by any convenient method. Advantageously, the protein of interest is obtained by expressing a recombinant vector according to the present invention or by culturing a cell according to the invention.

The protein of interest may be any protein for which structural data is desired. Preferably, the protein of interest is one according to the definition of the “second protein” given above. More preferably, the protein of interest is an integral membrane protein.

Typically, the x-ray diffraction data is obtained to a level of resolution which can yield structural information. This resolution may vary according to the detail of structural information required. Preferably, the resolution is at least 6 Å, more preferably at least 5 Å, still more preferably at least 4, 3.5, 3.2, or 3, or 2.5 Å.

The present invention further provides a use of a recombinant vector, or a cell, according to the invention, in a method of obtaining structural data on a protein of interest according to the invention.

In yet a further aspect, the invention provides a process for the production of a recombinant vector according to the invention comprising

(i) obtaining a recombinant vector comprising (I) a nucleotide sequence encoding a first protein which is a membrane protein or multisubunit protein and which, when crystallized with a second protein, is capable of accommodating the second protein in the crystal lattice and (II) a promoter operably linked to the said nucleotide sequence; and

(ii) introducing, into the said vector, nucleotide sequences facilitating the insertion of further nucleotide sequences wherein the resulting crystal would be capable of diffracting x-rays.

The nucleotide sequences may be sequences including a restriction endonuclease cleavage site.

An additional aspect of the invention provides a process for the production of a recombinant vector according to the invention, comprising

(i) obtaining a recombinant vector comprising (1) a nucleotide sequence encoding a first protein which is a membrane protein or multisubunit protein and which upon crystallization yields crystals having available space in the lattice, so as to allow for the ordered packing of a second protein into the said available space, and (II) a promoter operably linked to the said nucleotide sequence; and

(ii) introducing into the said vector, nucleotide sequences facilitating the insertion of further nucleotide sequences wherein the resulting crystal would be capable of diffracting x-rays.

The further nucleotide sequences are preferably sequences encoding at least one farther protein which is the protein to be crystallized.

The said recombinant vector obtained in step (i) could e.g. be the vector designated pMB908 (disclosed as “pJRhisA” by Rumbley et al. (1997) Biochimica et Biophysica Acta 1340, 131-142 which comprises the nucleotide sequence shown as SEQ ID NO: 1.

The nucleotide sequences may be sequences including a restriction endonuclease cleavage site.

### Experimental Methods

The modified vector constructs were generated using standard methods, such as molecular cloning methods, PCR, restriction analysis, DNA preparative methods, etc. In this context, the term “standard methods” is to be understood as referring to protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

Large-scale production of the cytochrome bo3 fusion protein was performed in E. coli, using a 1OL-fermentor. E. coli cells were grown in LB supplemented with 1 g KH2PO₄, 14.6 g K2HP₄, 20 ml Na-lactate, 0.5 ml 1 M MgSO₄, 0.00214 g CuSO₄, 0.0102g vitamin B1, 0.0098 g Nicotinic Acid and 0.05 g ampicillin/L over a period of 8 hrs (Georgiou et al. (1988) Biochim. Biophys. Acta 933,179-183).

### EXAMPLES OF THE INVENTION

### EXAMPLE 1

### Crystallization and Structural Determination of Cytochrome bo3

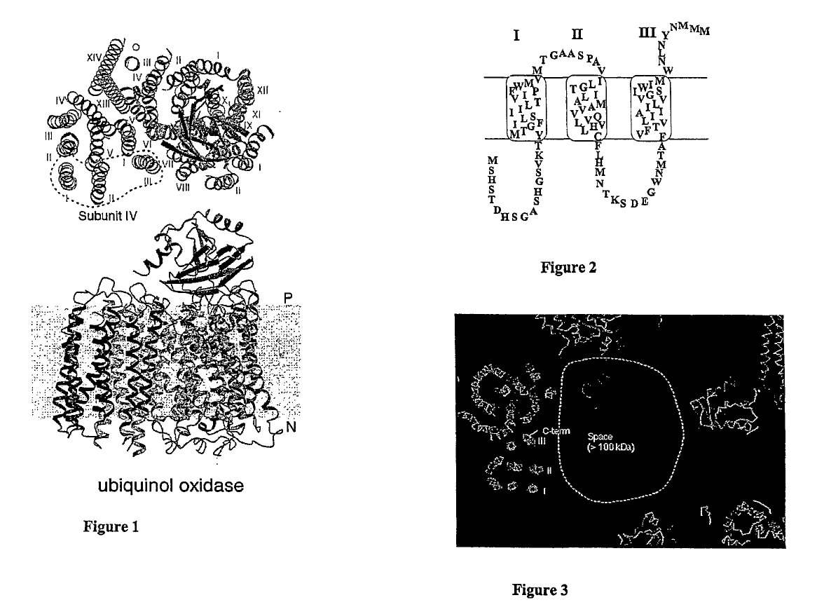

The structure of cytochrome bo3 from Eschezichia coli was determined. Plasmids encoding a carboxy-terminus histidine-tag on subunit II of cytochrome bo3 ubiquinol oxidase were cloned into an E. coli strain, G0105, lacking terminal oxidases as described in Kaysser et al. (1995) Biochemistry 34, 13491-13501. Purified protein was crystallized using polyethylene glycol 1500. Data collection from the crystal was performed at ID14/EH3 of the ESRF. Image data was processed up to 3.5 Åresolution (FIG.1) with an R_{merge} value of 10.8% (for F>1.0 (F)). The crystals belong to the space group C2221 with unit cell dimensions of a=92.1 Å, b=372.5 Å and c=232.7 Å. The asymmetric unit contained two molecules of ubiquinol oxidase. The bo3 protein has a molecular weight of 144 kDa and occupies about 41% of the volume of the unit cell.

EXAMPLE 2

### Expression Vector Constructs

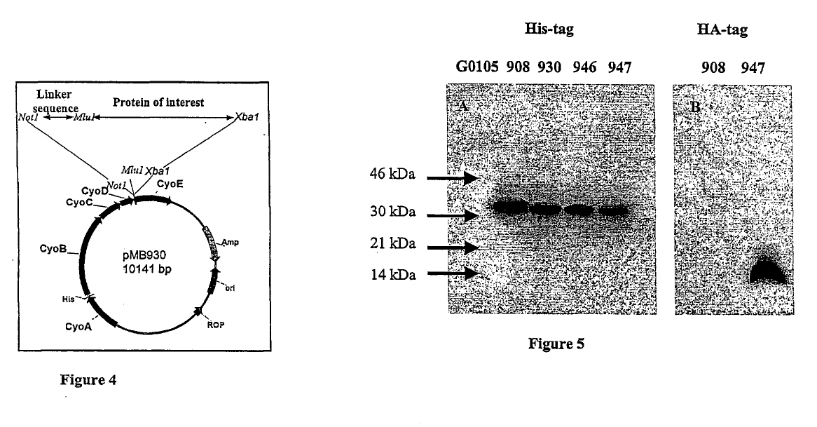

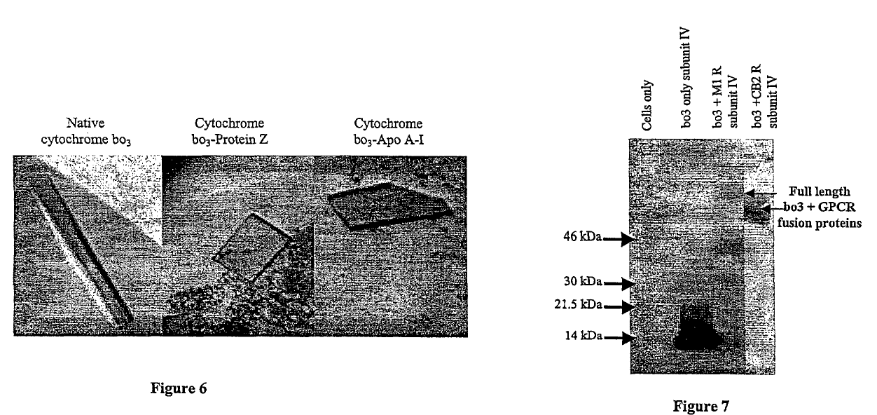

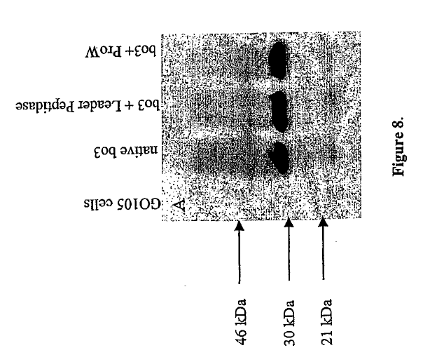

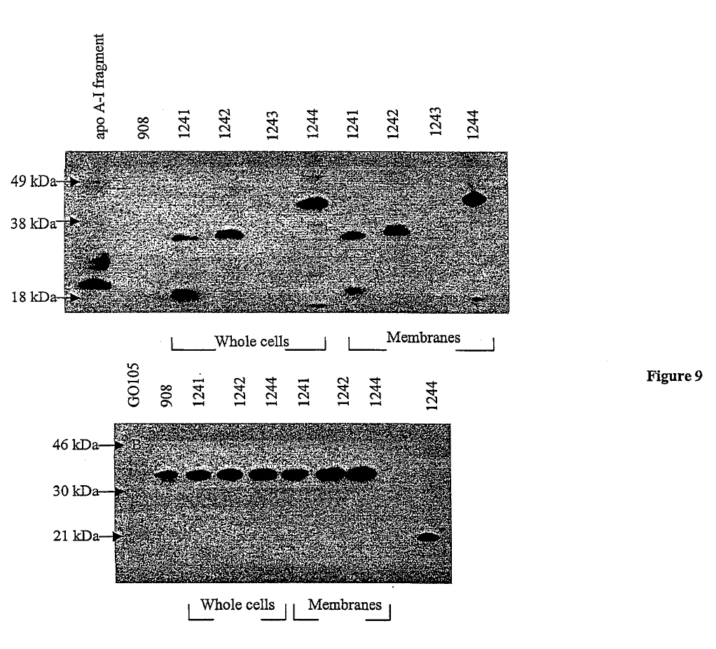

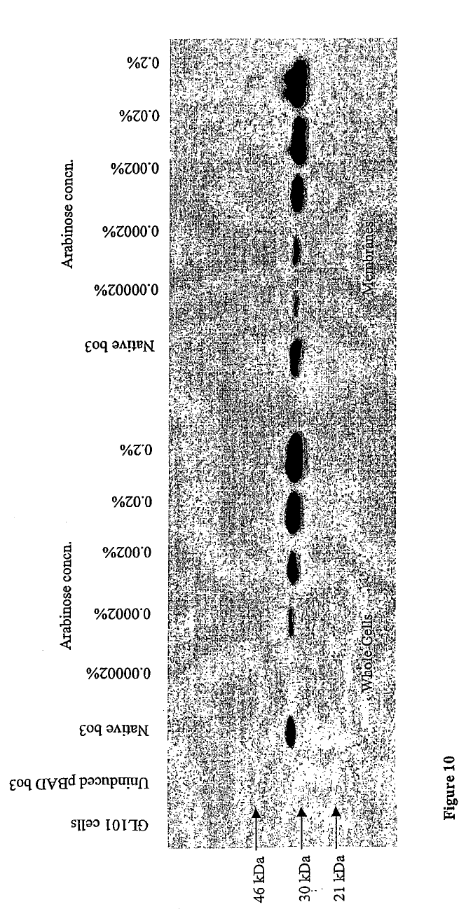

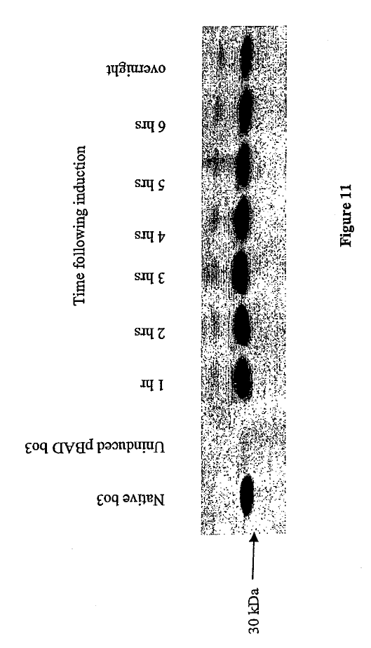

A multiple cloning site (NotI, SacI, MluI and XbaI) was added to the 5′-end of subunit IV of the cytochrome bo3 scene. A plasmid designated pMB908 (SEQ ID NO:1), which comprises a cytochrome bo3 construct with a His9 tag at the C-terminus of subunit II was used as starting material. The plasmid pMB908 is identical to the pJRhisA plasmid described by Rumbley et al. (1997) Biochimica et Biophysica Acta 1340, 131-142. The addition of unique restriction sites to the carboxy-terminus to subunit IV in pMB908 was performed by the polymerase chain reaction (PCR) method of splicing by overlap extension. The method entails the use of four different primers, two encompassing the entire sequence to be changed, one on either end of the new construct (the 5′-primer is set forth as SEQ ID NO: 2 and the 3′-primer as SEQ ID NO: 3), and two centrally positioned primers (The 5′-primer is set forth as SEQ ID NO: 4 and the 3′-primer as SEQ ID NO: 5) containing the sequence for the restriction sites (NotI, SacI, MluI, XbaI) giving rise to two fragments containing overlapping sequences. The PCR reactions are carried out in two steps to yield a 2.4 kb fragment of the cytochrome bo3 gene which was flanked by two unique restrictions sites (NsiI and SphI). This fragment was sequenced and then heated into the cytochrome bo3 gene generating the construct MB930 (FIG. 4).

Two sets of linkers were generated to act as bridge sequences between subunit IV and the foreign fusion protein. A short olioonucleotide linker, coding for the Strep-tag, a nonapeptide having the sequence AWRHPQFGG (SEQ ID NO: 6) was formed by annealing two single-stranded, synthetic oligonucleotides (The forward-strand oligonucleotide is set forth as SEQ ID NO: 7 and the reverse strand oligonucleotide as SEQ ID NO: 8), coding for this sequence and containing a 5′-NotI site and 3′-MluI site. The Strep-tag is an amino acid sequence which was identified using phage display based on its affinity for streptavidin (Schmidt & Skerra (1993) Protein Engineering 6, 109-122). A second linker (Strep-HA-tag; AWRHPQFGGYPYDVPDYA) (SEQ ID NO: 9) coding for both the Strep-tag and the hemaglutinin (HA)-tag YPYDVPDYA (SEQ ID NO:10) (Kast et al. (1996) J Biol. Chem. 271(16), 9240-9248), was made by annealing, linear, single-stranded, synthetic oligonucleotides (The forward-strand oligonucleotide is set forth as SEQ ID NO: 11 and the reverse-strand oligonucleotide as SEQ ID NO: 12). The oligonucleotide cassettes were generated by mixing 10 nmol of 5′-and 3′-oligonucleotide with 30 1 annealing buffer (500 MM NaCl, 100 nM Tris-HCI, pH 7.4, and 100 nM MgCl₂) in a total volume of 300 1. The samples were boiled for two minutes and then allowed to cool to approximately +30 C. prior to storage at −20 C. The linkers were cloned into the NotI/MluI sites of the construct pMB930, yielding constructs pMB946 and pMB947 coding for the Strep and Strep-HA linker sequences, respectively.

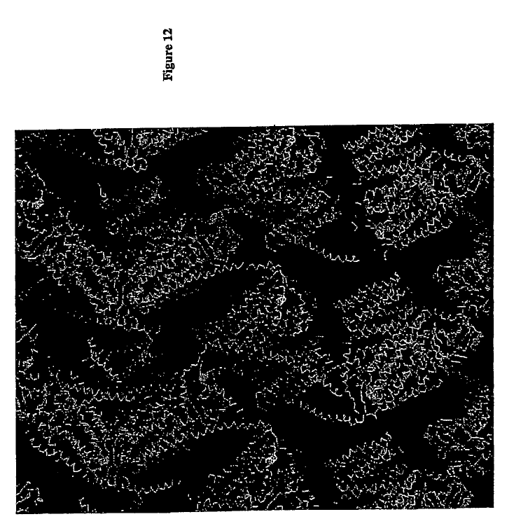

The expression of all the modified cytochrome bo3 constructs was assessed in two ways. Firstly, each construct was transformed into the E. coli strain G0105 Kaysser et al. (1995) Biochemistry 34:13491-13501. These cells lack an endogenous oxidase activity and will only grow under aerobic conditions after the introduction of a functional oxidase. All the vector constructs produced G0105 cell colonies, indicating that the additions to the sequence had not altered the function of the cytochrome bo3. Secondly, a His-tag was present at the C-terminal end of subunit II of the cytochrome bo3, and in pMB947 there was a HA-tag at the C-terminal end of subunit IV. It was thus possible -to assess the expression of the constructs using Western blot analysis (FIG. 5) with antibodies directed against these specific sequences.

### EXAMPLE 3

### Cytoclrome bo3 -Protein Z Fusion Protein

The polypeptide designated “Protein Z” or “Domain Z” (Nilsson et al. (1987) Prot. Eng., 107-13; SEQ ID NO: 14) is a modified analogue of the IgG-binding domain B of Staphylococcus aureus protein A (SPA). Protein Z (6.6 kDa) has been extensively used as an affinity tag for reviews see Nilsson et al. (1992) Curr. Opin. Struct. Biol. 2: 5 69575; and LaVallie McCoy (1995) Curr. Opin. Biotech 6: 501-506. The structure of SPA domain B has been resolved to 2.8 Å (Deisenhofer (1981) Biochemistry 20, 236170).

Protein Z was cloned into the NotI and MluI sites of the cytochrome bo3 fusion vector using standard methods yielding pMB1048. Expression of the cytochrome bo3 fusion construct was assessed by Western blot analysis and cells expressing the fusion protein were grown in a fermentor (OD₅₅₀=3.0), harvested and stored at −80 C. Membranes were purified by the following method: Cells from a 1OL culture were taken to a final volume of 1L in lysozyme treatment buffer (200 mM Tris-HCl, pH 8.8, 20 mM EDTA, pH 8.0, and 500 mM sucrose), lysozyme was added to a final concentration of 0.1% and the cells stirred for 30 min. The cells were pelleted by centrifugation at 8,000 rpm for 20 min. The pellets were resuspended in approx. 750 ml cell disruption buffer (5 mM EDTA, pH 8.0, 10 M PMSF, 10 mM MgCl₂ and several crystals of DNAse ) and stirred on ice for 15 min. The solution was sonicated on burst mode for 2×3 min. Unbroken cells were separated by centrifugation at 6000 rpm for 20 min and the supernatant centrifuged 45,000 rpm for 1 h. The membrane pellets were resuspended in a minimal volume of buffer (20 mM Tris-HCI, pH 7.5, 300 mM NaCl and 2.5 mM imidazole). At this point it was possible to freeze the membrane pellets at −80C. prior to solubilization and purification.

The membranes, were solubilized in 1% dodecylmaltoside for 1 h, +4 C. Solubilized protein was harvested after ultracentrifugation at 100,000×g for 1 h and applied to a 65 ml bed volume Ni-NTA column (Qiagen) equilibrated with 20 mM Tris-HCI, pH 7.5, 300 mM NaCl, 5 mM imidazole and 0.03% dodecylmaltoside. The column was washed with three bed volumes of the equilibration buffer to remove any non-specific binding proteins and then the sample was eluted with a linear 5-150 mM imidazole gradient. The resulting chromatograms showed two clear protein peaks, which were termed “low” and “high” imidazole based on the elution concentration.

The fractions from the two peaks were pooled separately. The buffer was then exchainged for 20 mM Tris-HCI, pH 7.5 containing 0.03% dodecyl maltoside using an Amicon stirred ultrafiltration cell (100 kDa cutoff filter) (Amicon, MA, USA). The two pools were then applied separately to an anion exchange column, MonoQ 10/10 (Amersham Pharmacia Biotech, Sweden) in the presence of 1 mM potassium ferricyanide after equilibrating the column with 20 mM Tris-HCl, pH 7.5 containing 1% octylglucoside. The column was washed slowly with 6 bed volumes of equilibration buffer and then eluted with a 0-600 mM NaCl gradient. Fractions containing the fusion protein were pooled on the basis of spectrophotometric readings. Using a Centricon ultrafiltration cell (100 kDa cutoff) (Millipore, MA, USA), the buffer was exchanged for 20 mM Tris-HCI, pH 7.5 containing, 1% octylglucoside, and the sample was concentrated to 20 mg protein/ml.

Crystals for this fusion protein (FIG. 6) were obtained for “low” and “high” imidazole protein, using the hanging drop vapor diffusion technique. The protein solution contained 20 mM Tris-HCI, pH 7.5, and 1% octylglucoside. A reservoir solution of 9-10% PEG 1500, 100 mM NaCl, 100 mM MgCl₂ and 5% ethanol was used. The protein solution was mixed in a 1:1 ratio with the reservoir solution and left to equilibrate at +4 C. These data demonstrate that the site at the C-terminal end of subunit IV can be used to accommodate foreign proteins as fusion partners without inhibiting crystal formation. Crystals of cytochrome bo3+Z, were obtained under similar conditions to the native cytochrome bo3 (Abramson et al, 2000), although the crystals of the fusion protein grew over a wider pH range (6-8) compared to the native protein (pH 7-7.5). In addition it was possible to grow crystals using protein from both the “low” and “high” imidazole peaks, compared to the native protein which only yielded reproducible crystals from the “low” imidazole peak. The crystals of the fusion protein from both “low” and “high” imidazole peaks grow as square plates, a more regular shape compared to the rod-like crystals of the native cytochrome bo3. Data was collected from a cytochrome bo₃+protein Z fusion protein crystal which diffracted X-ray up to 6 Å and had a space group P2₁ with the cell dimensions a=93.4 Å, b=328.7 Å, c=131.1 Å and *=92.1 with four molecules in the assymetric unit. These data are in contrast with the wild-type protein crystals which have a space group C222₁ with the cell dimensions a=91.3 Å, b=370.3 Å, c=232.4 Å (see example 1).

### EXAMPLE 4

### Cytochrome bo3—GPCR Fusion Proteins

Two G-protein coupled receptors (GPCRs); the human muscarinic 1 (M1) receptor of 51 kDa (Allard et al. (1987) Nucleic Acids Res 15: 10604) and the human canabinoid 2 (CB2) receptor of 40 kDa (Munro et al. (1993) Nature 365, 61-65) were cloned into the MluI and XbaI sites of the cytochrome bo3 fusion vectors according to standard methods. The receptors expressed as fusion partners with subunit IV of the cytochrome bo3 (FIG. 7).

### EXAMPLE 5

### Cytochrome bo3—Leader peptidase fusion protein and cytochrome bo3 ProW fusion protein.

A fusion construct of cytochrome bo3+E. coli leader peptidase (Whitchurch & Mattick (1994) Gene 150: 9-15) (no tag sequence in vector) was obtained as described above. The biological role of the E. coli leader peptidase (36 kDa) is to remove amino-terminal leader peptides from exported proteins after they have crossed the plasma membrane. The enzyme of 323 amino acid residues spans the membrane twice, with its large carboxy-terminal domain protruding into the periplasm (for a review, see Dalbey (1991) Mol. Microbiol. 5, 2855-2860).

A fusion construct of cytochrome bo3+E coli ProW (Growsbiker (1989) J. Bacteriol. 171: 1923-193 1) (+Strep-tag) was obtained as described above. ProW is an E. coli inner membrane protein of 38 kDa that consists of a 100-residue-long periplasmic N-terminal tail followed by seven closely spaced transmembrane segments (Cristobal et al. (1999) J. Biol Chem. 274, 20068-20070). It is part of the ProU system, a member of the ATP-binding cassette (ABC) superfamily of transporters (Lucht & Bremer (1994) FEMS Microbiology Letters 14: 3-20). Both fusion constructs were shown to express (FIG. 8).

### EXAMPLE 6

### Cytochrome bo3—Apo AI Fusion Protein

Apolipoprotein AI (Apo AI, Sharpe et al. (1984) Nucleic Acids Res. 12: 3917-3932) is the major protein component (28 kDa) of the serum high-density lipoprotein (HDL) particles (for a review, see Hargrove et al. (1999) J Mol. Endocrinol. 22, 103-111). The structure of truncated human Apo AI has been determined at 3 Å resolution (Borhani et al. (1999) Acta Cryst. D 55: 12291-12296).

The following fusion constructs of cytochrome bo3 and Apo AI were generated:

pMB1241 Cytochrome bo3+Apo AI (no tag)

pMB1242 Cytochrome bo3+Strep-tag+Apo AI

pMB1243 Cytochrome bo3+Strep-HA-tag+Apo AI

pMB 1244 Cytochrome bo3+Protein Z+Apo AI

Interestingly, the expression of these fusion proteins was different from the earlier expressed proteins (FIG. 9). No expression of cytochrome bo3+Strep-HA-tag+Apo AI (pMB1243) could be detected. The cytochrome bo3+Protein Z +Apo AI (pMB1244) exhibited the highest level of expression, although it underwent a certain amount of proteolytic degradation. Cytochrome bo3+Apo AI (no tag) exhibited detectable levels of expression (pMB1241) although it too degrades (50% loss). The cytochrome bo3+Strep-tag+Apo AI (pMB1242) construct expresses at relatively high levels and does not appear to be degraded. In addition, the fusion proteins only appear to express satisfactorily to an OD600 of approximately 1, after which the whole cytochrome bo3 Apo AI complex appears to be broken down. These results show the individuality of proteins expressed in this system and highlight the need to characterize the growth and production of each fusion protein. Optimizing the construct as well as the host system and cultivation conditions with regard to the properties of the target protein may be performed by a person skilled in the art as shown in the current example.

It was possible to obtain crystals of pMB1242, cytochrome bo3+apo A-I. These had an elongated hexagonal plate form. In addition, these crystals were also thicker and had sharper edges than the native crystals, indicating ordered packing within the crystal lattice. These crystals diffracted to 5 Å and belong to the space group C2 with the cell dimensions a=93.4 Å, b=328.7 Å, c=131.1 Å and *=92.1 and two molecules per assymmetric unit. Crystals of pMB1246, cytochrome bo3+ProW, were obtained under slightly different conditions; PEG 1500 concentration was 18% and pH 6.5. These crystals diffracted to 6 Å and belong to the space group C2 with the cell dimensions a=93.4 Å, b=328.7 Å, c=131.1 Å and *=92.1 and two molecules per assymmetric unit. The wide-belt like loop of the apolipoprotein circles the whole cytochrome bo3 molecule and forms protein-protein contacts within the lattice. It is possible that given the function of apo A-I in vivo, it is binding to the detergent micelle which surrounds the hydrophobic portions of cytochrome bo3. The apolipoprotein A-I appears to be accommodated within multiple gaps in the crystal lattice which are connected by solvent channels.

### EXAMPLE 7

### Expression of Cytochrome bo3 Under the Control of an Inducible Promoter.

The constitutive cytochrome bo3 promoter was replaced with an inducible pBAD promoter cloned using the pBADHis vector (Invitrogen Corp., CA, USA) as a template. The pBAD Expression System is based on the araBAD operon, which controls the arabinose metabolic pathway in E. coli. This construct was expressed in an alternative cell line, GL1O1 (Rumbley et al. (1997) Biochem. Biophys. Acta 1340: 131-142). These cells express a form of oxidase and thus will grow under aerobic conditions in the absence of bo3. The expression of cytochrome bo3 was induced with increasing concentrations of arabinose, once the cells reached an OD600 of 0.5. Maximum expression of the cytochrome bo3 construct was observed at 0.2% arabinose with higher concentrations causing no further significant increase in expression.

The expression of cytochrome bo3 under control of the inducible promoter (pMB1127) was higher than that under control of the constitutive promoter (pMB908) (FIG. 10). Time course studies showed that the maximum detectable expression took place within 3-4 hours post induction (FIG. 11).

pBAD expression vectors were (generated for cytochrome bo3 with the MCS, the Streptag, the Strep-HA-tag and protein Z yielding plasmids pMB1271, pMB1128, pMB1272, pMB1270 respectively. Nucleotide sequences coding for polypeptides such as e.g. Apo AI or the CB2 receptor can be cloned into these vectors, and the expression of such polypeptides under control of the inducible promoter can be determined.

### EXAMPLE 8

### Use of Fumarate Reductase as a Scaffold Molecule

The E. coli respiratory enzyme fumarate reductase (FRD) is a four-subunit protein with a molecular mass of 121 kDa, which catalyzes fumarate reduction to succinate using membrane-bound menaquinol in anaerobic respiration (Kroger (1978) Biochim. Biophys. Acta 505, 129-145; Cole et al. (1985) Biochim. Biophys. Acta 811, 381-403).

Recently, the structure of FRD has been solved to 3.3 Å (Iverson et al. (1999) Science 284, 1961-1966) and subsequently to 2.8 Å (Tina Iverson, personal communication). In similarity with cytochrome bo3, the crystal lattice of FRD incorporates a gap, which could be exploited for crystallization of heterologous proteins. Two subunits of FRD, FrdC and FrdD, have three transmembrane helices and it would be possible to make fusion proteins at the C-terminal of these subunits using similar constructs as those described for cytochrome bo3. The expression level of FRD is very high under anaerobic conditions and purification is facilitated by the fact that the protein is selectively solubilized by the nonionic detergent Thesit (Maklashina (1998) J. Bacteriol. 180, 5989-5896), also called Polidocanol. The same restriction sites at the 3′-ends of FrdC and FrdD as for cytochrome bo3 subunit IV are generated, to allow simple transfer of nucleic acid sequences encoding the target proteins between scaffold molecules.

1. A recombinant vector comprising, (i) a promoter sequence and (ii) a nucleotide sequence encoding a first protein which is a membrane protein or multisubunit protein and which, when crystallized with a second protein, is capable of accommodating the second protein in the crystal lattice; said recombinant vector further allowing for the insertion of a further nucleotide sequence encoding a second protein to be located, when crystallized, in the crystal lattice of the first protein wherein the resulting crystal lattice is capable of diffracting x-rays.

2. A recombinant vector comprising, (i) a promoter sequence and (ii) a nucleotide sequence encoding a first protein which is a membrane protein or multisubunit protein and which upon crystallization yields crystals having available space in the lattice, so as to allow for the ordered packing of a second protein into the said available space; said recombinant vector further allowing for the insertion of a further nucleotide sequence encoding a second protein to be accommodated, upon its crystallization, in the said available space in the lattice of the first protein wherein the resulting crystal lattice is capable of diffracting x-rays.

3. A recombinant vector according to claim 1 or 2 wherein the x-ray diffraction is to a resolution of at least 5 Å.

4. A recombinant vector according to claim 3 wherein the diffraction resolution is at least 4 Å.

5. A recombinant vector according to any one of claims 1 to 4 wherein the first protein is a fusion partner of the second protein.

6. A recombinant vector according to claims 1 to 5 wherein the crystal space group of the first protein when crystallised alone may be different to that obtained by crystallisation with the second protein.

7. A recombinant vector according to any one of claims 1 to 6 wherein the said nucleotide sequence encoding a first protein is a sequence encoding a multisubunit protein.

8. The recombinant vector according to any one of claims 1 to 7 wherein the said nucleotide sequence encoding a first protein is a sequence encoding a membrane protein.

9. The recombinant vector according to any one of claims 1 to 8 wherein the said nucleotide sequence encoding a first protein is a sequence encoding an integral membrane protein.

10. The recombinant vector according to claim 9 wherein the integral membrane protein has one transmembrane domain.

11. The recombinant vector according to any one of claims 1 to 10 wherein the size of the first protein encoded by the nucleotide sequence is more than 10 amino acids in total.

12. The recombinant vector according to any one of claims 1 to 10 wherein the said nucleotide sequence encoding a first protein is a sequence encoding E. coli cytochrome bo3 or E. coli fumarate reductase, or variants thereof.

13. The recombinant vector according to claim 12 wherein the said nucleotide sequence encoding a first protein is a sequence encoding E. coli cytochrome bo3 or a variant thereof.

14. The recombinant vector according to claim 13 wherein the said nucleotide sequence encoding E. coli cytochrome bo3 is selected from
(a) the polypeptide coding regions of the nucleotide sequence shown as SEQ ID NO: 13;
(b) nucleotide sequences capable of hybridizing under stringent hybridization conditions, to a nucleotide sequence complementary with the polypeptide coding regions of the nucleotide sequence as defined in
(a), and
(c) nucleic acid sequences which are degenerate as a result of the genetic code to a nucleotide sequence as defined in (a) or (b).

15. The recombinant vector according to claim 13 or 14 wherein the said promoter sequence essentially comprises the cytochrome bo3 promoter sequence shown as positions 203 through 803 in SEQ NO: 1.

16. The recombinant vector according to any one of claims 1 to 15 wherein the said promoter is an inducible promoter.

17. The recombinant vector according to any one of claims 1 to 16, further comprising a nucleotide sequence encoding a linker amino acid sequence facilitating for the said first and second proteins to be expressed as a fusion protein.

18. The recombinant vector according to claim 17 wherein the said linker amino acid sequence is adapted to facilitate, upon expression of the said first and second proteins, for the said second protein to be positioned in the said available space in the crystal lattice of the first protein.

19. The recombinant vector according to claim 17 or 18 wherein said linker amino acid sequence is a Strep-tag having an amino acid sequence shown as SEQ ID NO: 6.

20. The recombinant vector according to claim 17 or 18 wherein said linker amino acid sequence is a Strep-HA-tag having an amino acid sequence shown as SEQ ID NO: 9.

21. The recombinant vector according to any one of claims 17 to 19 wherein the said nucleotide sequence coding for a linker amino acid sequence is positioned at the 3′-end of the nucleotide sequence coding for E. coli cytochrome bo3 subunit IV.

22. The recombinant vector according to any one of claims 1 to 21 in addition comprising a nucleotide sequence encoding a polypeptide having essentially an amino acid sequence shown as SEQ ID NO: 14.

23. The recombinant vector according, to any one of claims 1 to 22, in addition comprising a nucleotide sequence encoding an affinity tag.

24. The recombinant vector according to claim 23, wherein the said affinity tag is a His-tag.

25. The recombinant vector according to claim 23 or 24, wherein the said first protein is E. coli cytochrome bo3 and wherein a nucleotide sequence encoding an affinity tag is attached to the nucleotide sequence encoding E. coli cytochrome bo3 subunit II.

26. The recombinant vector according to any one of claims 1 to 25, further comprising a nucleotide sequence encoding the said second protein.

27. The recombinant vector according to claim 26, wherein the said second protein has a molecular mass below 100 kDa.

28. The recombinant vector according to claim 26 or 27 wherein the second protein has a lower molecular weight than the first protein.

29. The recombinant vector according to any one of claims 22 to 28 wherein the said second protein is a membrane protein.

30. A cultured host cell harbouring a recombinant vector as defined in any one of claims 26 to 29.

31. The host cell according to claim 30 which is an E. coli cell.

32. A process for the production of a fusion protein which comprises culturing a host cell as defined in claim 30 or 31 under conditions whereby the said fusion protein is produced, and recovering the said fusion protein.

33. A fusion protein obtained or obtainable by the process as defined in claim 32.

34. A fusion protein comprising (i) a first protein which is a membrane protein or multisubunit protein and which upon crystallization yields crystals having available space in the lattice, so as to allow for the ordered packing of a second protein into the said available space; and
(ii) a second protein to be accommodated, upon crystallization, in the said available space wherein the resulting crystal is capable of diffracting x-rays.

35. A fusion protein comprising (i) a first protein which a first protein which is a membrane protein or multisubunit protein and which, when crystallized with a second protein, is capable of accommodating the second protein in the crystal lattice and (ii) a second protein to be located, when crystallized, in the crystal lattice of the first protein wherein the resulting crystal lattice is capable of diffracting x-rays.

36. A fusion protein according to claim 34 or 35 wherein either or both of the first and second proteins are integral membrane proteins.

37. The fusion protein according to claim 32 to 36 wherein the said first protein is E. coli cytochrome bo3.

38. The fusion protein according to claim 37 wherein the said second protein is attached to subunit IV of E. coli cytochrome bo3.

39. A method for crystallization of a protein, comprising
(i) obtaining a fusion protein comprising (a) a first protein, which is a membrane protein or multisubunit protein and which upon crystallization yields crystals having available space in the lattice, so as to facilitate crystallization of a second protein; and (II) the said (second) protein to be crystallized; and
(ii) crystallizing the said fusion protein
wherein the resulting crystal is capable of diffracting x-rays.

40. A method for crystallization of a protein, comprising
(i) obtaining according to the process as defined in claim 32, a fusion protein; and
(ii) crystallizing the said fusion protein
wherein the resulting crystal is capable of diffracting x-rays.

41. A method for crystallization of a protein, comprising
(i) obtaining a fusion protein as defined in any one of claims 33 to 38; and
(ii) crystallizing the said fusion protein.

42. A method according to any one of claims 39 to 41 wherein the first protein is an integral membrane protein.

43. A method for crystallization of a protein, comprising
(i) obtaining a first protein which is an integral membrane protein and which upon crystallization yields crystals having available space in the lattice so as to facilitate crystallization of a second protein; and
(ii) obtaining the second protein to be crystallized; and
(iii) crystallizing both the said proteins together
wherein the resulting crystal is capable of diffracting x-rays.

44. A method according to claim 43 wherein the second protein is soaked into a crystal of the first protein.

45. A method according to any one of claims 39 to 44 wherein the first protein is as defined in any one of claims 1 to 24.

46. A method according to any one of claims 39 to 45 further comprising a step wherein at least two detergents are screened in the crystal growth conditions to identify which one optimizes the growth and/or diffraction of the resulting crystals.

47. A method according to any one of claims 39 to 46 further comprising a step wherein the pH is optimized for crystal growth.

48. A method according to any one of claims 39 to 47 wherein the crystal space group of the first protein when crystallized alone may be different to that obtained by crystallization with the second protein.

49. A method according to any one of claims 39 to 48 wherein the second protein is an integral membrane protein.

50. A method according to any one of claims 39 to 49 wherein the second protein has a lower molecular weight than the first protein.

51. A method of obtaining structural data on a protein of interest comprising the steps of
(i) obtaining the protein of interest;
(ii) crystallising said protein in the crystal lattice of another protein, which crystal lattice is able to accommodate the protein of interest; and
(iii) obtaining x-ray diffraction data from the crystal produced in step (ii).

52. A method according to claim 51 wherein the crystallisation method is according to any one of claims 39 to 49.

53. A method according to claim 51 or 52 wherein the protein of interest is obtained by expressing a recombinant vector according to any one of claims 26 to 29 or by culturing a cell according to claim 30.

54. A method according to any one of claims 51 to 53 wherein the protein of interest is an integral membrane protein.

55. A method according to any one of claims 51 to 53 wherein the x-ray diffraction data is obtained to a resolution of at least 6 Å.

56. Use of a recombinant vector according to any one of claims 27 to 29 or a cell according to claim 30 in a method according to any one of claims 51 to 55.

57. A process for the production of a recombinant vector according to claim 1 comprising
(i) obtaining a recombinant vector comprising (I) a nucleotide sequence encoding a first protein which is a membrane protein or multisubunit protein and which, when crystallized with a second protein, is capable of accommodating the second protein in the crystal lattice and (II) a promoter operably linked to the said nucleotide sequence; and
(ii) introducing, into the said vector, nucleotide sequences facilitating the insertion of further nucleotide sequences
wherein the resulting crystal would be capable of diffracting x-rays.

58. A process for the production of a recombinant vector according to claim 3, comprising
(i) obtaining a recombinant vector comprising (I) a nucleotide sequence encoding a first protein which is a membrane protein or multisubunit protein and which upon crystallization yields crystals having available space in the lattice, so as to allow for the ordered packing of a second protein into the said available space, and (II) a promoter operably linked to the said nucleotide sequence; and
(ii) introducing, into the said vector, nucleotide sequences facilitating the insertion of further nucleotide sequences
wherein the resulting crystal would be capable of diffracting x-rays.

59. The process according to claim 57 or 58 wherein the said recombinant vector obtained in step (i) comprises the nucleotide sequence shown as SEQ ID NO: 1
